(19) United States
(12) Patent Application Publication
(10) Pub. No.: US 2008/0206310 A1
(43) Pub. Date: Aug. 28, 2008

(54) Regimens for Controlled Drug Delivery Devices for Contraception

(75) Inventor: Victoria Jane Davis, Toronto (CA)

Correspondence Address: ORGANON USA, INC.;c/o Schering-Plough Corporation, 2000 Galloping Hill Road, Mail Stop: K-6-1, 1990, Kenilworth, NJ, 07033, US

(73) Assignee: N.V. Oraganon, Oss (NL)

(21) Appl. No.: 11/917,485

(22) PCT Filed: Jun. 16, 2006
(86) PCT No.: PCT/US2006/023383

§ 371 Date: Dec. 13, 2007

(30) Foreign Application Priority Data
Jun. 21, 2005 (EP) 05105484.9

(51) Int. Cl. A61F 6/14; A61K 31/57; A61P 15/18

(52) U.S. Cl. 424/432; 514/170

(57) ABSTRACT

The subject invention provides for new regimens for contraceptive dosage forms.

The present invention relates mainly to the field of female reproductive medicine, and in particular to human female contraception. The present invention relates to new regimens for administration of controlled drug delivery devices, e.g. to achieve contraception or to treat and/or prevent other hormone cycle-dependent indications such as dysmenorprhoea, menorrhagia, irregular menstruation, menstrual migraine and premenstrual syndrome (PMS).

It is standard practice in the field of oral contraceptive regimes that the definition of the cycle duration is linked to a fixed number of days and weeks. These kind of regimens evolved due to the need to mimic the menstrual cycle in the development of contraceptives. As a result thereof, women start a new cycle of contraception at a fixed day of a week, e.g. a Sunday, a Monday etc. Another result thereof is that patient packs with identical content and form (such as oral contraceptive strips with 21 or 28 pills) can be produced which in turn ensures economic production methods and helps the user to acquire habits needed for consistent self-administration of the tablets for contraception.

With evolving technology and new methods of drug delivery, the cycle is no longer determined by daily intake of active ingredients but by determinants of the drug delivery device or system itself.

Controlled drug delivery devices are known in the art. For example, vaginal rings, implants, patches, hormonal intra-uterine devices (IUDs), spray devices, etc. used for achieving contraception are known in the art. For example a vaginal ring is a controlled drug delivery device for a complete cycle.

EP 876 815 discloses the only commercially available vaginal ring (Nuvaring®) for contraception which is designed for the simultaneous release of a progestogenic steroid compound and an estrogenic steroid compound.

Nuvaring comprises 11.7 mg etonogestrel (released at a rate of 0.120 mg per 24 hours), 2.7 mg ethinyl estradiol (released at a rate of 0.015 mg per 24 hours) and ethylene vinyl acetate copolymer.

The current regimen for this vaginal ring is that it is inserted for 21 days and then removed for a period of one week (7 days) to permit vaginal bleeding. After the week to allow for vaginal bleeding, a new ring is inserted into the female vagina to provide contraception in the next female cyclus or cyclusses.

The disadvantage of such a regimen is that it requires a woman to remember many moments in time. For each ring used, a woman has to remember to remove the ring when 21 days are over from the day of insertion and then to remember to insert a new ring when 7 days are over. These crucial moments that have to be remembered fall on different dates each time. For example, if a woman removed a ring on January 1, then a new ring has to be inserted on January 8 and removed again on January 29. A new ring will then have to be inserted on February 5 which ring will then have to be removed on February 26 etc. etc. It is clear that it is difficult to keep track of these dates. As a result thereof some women forget to insert and/or remove their ring in a timely fashion resulting in unwanted pregnancies.

Even though devices are available to help women remember to start and remove their contraceptive (see e.g. EP 1257244), it would be desirable to have a regimen which maintains contraceptive effect and which also reminds a woman automatically of the crucial dates, thereby avoiding the risk of a woman forgetting to remove a ring (or other dosage form) or insert a new ring (or other dosage form) and becoming pregnant.

The subject invention now provides for new regimens for the administration of contraceptive dosage forms (and for the administration of dosage forms to treat and/or prevent other cycle-dependent indications), resulting in improved compliance while maintaining contraceptive efficacy. This improved compliance is enabled by the functional combination of at least two dosage forms as defined herein during at least two cycles as defined herein. Compliance is enabled with the administration of at least the second dosage form. Without any at least second dosage form one would not have to look at compliance. Therefore to obtain the effect of improved compliance, the functional combination of at least two dosage forms is a prerequisite.

The new regimens of the present invention further result in that women will have only 12 periods a year as opposed to thirteen in standard 21/7 regimens.

Controlled drug delivery devices do not impose the constraints to provide for a cycle of fixed duration. The present invention now exploits this new facility by providing regimens which are not constrained by identical cycles of fixed duration but enable flexible cycle duration. The subject invention has the important advantage to help user habit acquisition, because start and removal of a dosage form used in the regimen of the subject invention is enabled on fixed numerical days of the month. Thus, the invention provides for a contraceptive regime with cycles of hormone administration for defined cycle durations, such that the cycle durations vary in order to correspond with the number of days of the calendar month in which the cycle is starting.

Both “month” and “calendar month” as used herein means any month, i.e. January, February, March, April, May, June, July, August, September, October, November, or December.

A “numerical date” as used herein is any existent date of a month. For example, January has 31 numerical dates. January 1, January 2, January 3 etc. etc. February has 28 or 29 numerical dates; March has 31 numerical dates; April has 30 numerical dates, etc.

“Cycle” as used in the subject invention is the duration of the number of the days of the month in which the cycle is started. During a cycle there is a hormone taking phase (or period or interval) and a hormone free phase (or period or interval). For example, a cycle which is started in January is 31 days; a cycle which is started in February is 28 or 29 days depending on whether it is a leap year or not; a cycle which is started in March is 31 days; a cycle which is started in April is 30 days, etc, etc. In addition, a cycle of the subject invention is a partial circle of events wherein the hormone levels in a woman increase and decrease due to the use of the dosage form. In order to complete the circle of events wherein hormone levels increase, decrease, increase again and decrease again, a woman must complete at least two cycles of the dosage form.

“Starting” as used herein means applying or inserting or any other form of contraceptive or pharmaceutical administration. For example, patches are applied and rings are inserted.

“Removing” as used herein means removing or tearing or any other form of removal of a contraceptive or pharmaceutical dosage form. For example, a vaginal ring is removed and a patch is torn.

A dosage form as used herein means a controlled release drug delivery device such as a vaginal ring or a patch.

A patch (a transdermal system) as used herein can be any (contraceptive) patch of any type, e.g. a matrix type, a reservoir type, a patch with multiple layers or a patch in which the drug is present in the adhesive as long as the patch that is used has sufficient active ingredient(s) for at least one cycle of contraception.

The only commercially available contraceptive patch currently on the market is Evra®. The Evra® patch however contains active ingredients sufficient for only one week of contraception, i.e. not for an entire cycle as defined herein.

A vaginal ring as used herein can be any (contraceptive) vaginal ring such as Nuvaring® or vaginal rings such as described in WO 2004/103336, PCT/EP05/051189, U.S. Pat. No. 4,292,965, WO 97/02015, EP887074.

A dosage form useful in the subject invention may comprise an estrogen, a progestogen or combinations thereof. It may optionally also contain other active ingredients such as anti-microbials, folic acid, vitamins etc.

Progestogen as used herein can be any suitable progestogen, such as desogestrel, etonogestrel, levonorgestrel, norgestimate, norelgestromin, gestodene, nomegestrol acetate, dienogest, drospirenone, or any other steroidal or non-steroidal compound with progestogenic activity.

The estrogenic compound as used herein can be any suitable estrogen (or salt thereof or ester thereof), such as estradiol, estriol, mestranol and ethinyl-estradiol or any other steroidal or non-steroidal estrogen with estrogenic activity.

In a specific embodiment of the subject invention, the progestogen is etonogestrel. In another embodiment, the progestogen is nomegestrol acetate.

In one embodiment of the subject invention the estrogen is ethinyl estradiol. In another embodiment, the estrogen is estradiol or an ester thereof or a salt thereof, such as estradiol hemi-hydrate.

In a specific embodiment, the progestogen is etonogestrel and the estrogen is ethinyl estradiol or a salt thereof or an ester thereof.

In another specific embodiment, the progestogen is nomegestrol acetate and the estrogen is estradiol or a salt thereof or an ester thereof.

In a specific embodiment, the progestogen is etonogestrel and the estrogen is estradiol or a salt thereof or an ester thereof.

In another specific embodiment, the progestogen is nomegestrol acetate and the estrogen is ethinyl estradiol.

As used herein, both ‘non-hormonal phase’ and ‘hormone-free phase’ is a phase (or period or interval) during a cycle in which no hormones are administered.

As used herein, ‘hormonal phase’ is a phase (or period or interval) during a cycle in which hormones are administered.

A vaginal ring used in the subject invention may comprise one or more compartments. Each compartment may comprise one or more layers. Such vaginal ring can be made of any material suitable to make such dosage forms. For example, a polymer that can be used in practicing the invention may in principle be any thermoplastic polymer or elastomer material suitable for pharmaceutical use, such as low density polyethylene, ethylene-vinylacetate copolymers, polysiloxane, polyurethane, polyacrylate and styrene-butadiene-styrene copolymers. In a specific embodiment, ethylene-vinylacetate copolymer (poly-EVA) is used which is commercially available under e.g. the trade names: Elvax, Evatane, Lupolen, Movriton, Ultrathene, Ateva and Vestypar.

Thus, improved compliance is enabled by using a method of human female contraception wherein a dosage form is started on numerical date ‘n+3’ of any month and removed on numerical date ‘n’ of the following month wherein ‘n’ is the numerical date of a month from 1-25 independent of which month and wherein the method is repeatedly carried out for at least two cycles. When using this method, the non-hormonal phase is a period of no longer than 4 days.

This improved compliance is also enabled by using a method of human female contraception wherein a dosage form is started on numerical date ‘m+4’ of any month and removed on numerical date ‘m’ of the following month wherein ‘m’ is the numerical date of a month from 1-24 independent of which month and wherein the method is repeatedly carried out for at least two cycles. When using this method, the non-hormonal phase is a period of no longer than 5 days.

This improved compliance is further enabled by using a method of human female contraception wherein a dosage form is started on numerical date ‘y+5’ of any month and removed on numerical date ‘y’ of the following month wherein ‘y’ is the numerical date of a month from 1-23 independent of which month and wherein the method is repeatedly carried out for at least two cycles. When using this method, the non-hormonal phase is a period of no longer than 6 days.

This improved compliance is additionally enabled by using a method of female human contraception wherein a dosage form is started on numerical date ‘z+6’ of any month and removed on numerical date ‘z’ of the following month wherein ‘z’ is the numerical date of a month from 1-22 independent of which month and wherein the method is repeatedly carried out for at least two cycles. When using this method, the non-hormonal phase is a period of no longer than 7 days.

The method of the subject invention can be used for any number of cycles starting with at least two cycles, i.e. for two, three, four, five, six, etc. cycles. In a specific embodiment, the method is used for at least three cycles.

In the methods of contraception of the subject invention, the hormonal phase between months is not constant. The non-hormonal phase on the other hand is constant between calendar months. In spite thereof, in all embodiments envisaged by the subject invention, ovarian suppression (necessary to achieve contraception) is maintained and in certain cases even improved.

Compliance is thus enabled by the fact that a woman can choose a particular numerical date of the month which she finds an easy number to remember. On this day, the dosage form will always be removed. Contraceptive efficacy is maintained during the period of contraception independent of the fact that the hormonal phase is not constant between months, whereas the non-hormonal phase is constant between months.

Thus, for example in an (n, n+3) regimen, a woman can choose ‘n’ to be any numerical date between 1-25, independent of which month.

For example, the first of the month is mostly an easy number to remember. In that example, in a (n, n+3) regimen, a woman chooses ‘n’ to be 1, i.e. the first of the month. Then, the dosage form is inserted (started) each 4^{th} day of the month (‘n+3’ numerical date of the month). For example, the 4^{th} of January, the 4^{th} of February, the 4^{th} of March etc. etc. The dosage form is then removed on each first day of the month, for example, the 1^{st} of January, the 1^{st} of February, the 1^{st} of March etc. etc. The woman now thus only has to remember the same two numerical dates each month, namely the 1^{st} and the 4^{th} independent of the month. Although not limiting the subject invention thereto, assuming that the dosage form is started on the fourth day of the month at the same time as it has been removed on the first day of the month, the duration of the hormonal phase is:

25 days in February

26 days in February of a leap year

27 days in April, June, September, November

28 days in January, March, May, July, August, October, December

Within the same assumption, the duration of the hormone free phase is constant and lasts 3 days. If the time of the day of insertion (start) is not the same time of the day of removal then the hormone free phase can be longer up to a maximum of 4 days when e.g. the woman removes the dosage form at 00.01 hours on the first of the month and inserts (starts) a new dosage form at 23.59 hours on the fourth day of the month. Thus, in the subject example (n, n+3) regimen, the hormone free interval is between 3-4 days but not longer than 4 days.

For example, when looking at a complete (non-leap) year starting in January and assuming that the dosage form is inserted on the fourth day of each month at the same time as it has been removed on the first day of the following month, then this (n, n+3) regimen each month has a hormone free-period of 3 days and hormone administration days as follows:

| January | February | March | April | May | June | July | August | September | October | November | December |
|---|---|---|---|---|---|---|---|---|---|---|---|
| 28 | 25 | 28 | 27 | 28 | 27 | 28 | 28 | 27 | 28 | 27 | 28 |

The concept is similar for the (m, m+4), (y, y+5) and (z, z+6) regimens. In a (m, m+4) regimen, the hormone-free phase is at least 4 days but no longer than 5 days, in a (y, y+5) regimen, the hormone free phase is at least 5 days but no longer than 6 days and in a (z, z+6) regimen, the hormone-free phase is at least 6 days but no longer than 7 days.

A regimen of the subject invention has at least two advantages: First of all, compliance is enabled because it is much easier to remember, for a woman using a particular dosage form, that on a particular day of any month she has to remove (stop) the dosage form and 3, 4, 5 or 6 days later (depending on the regimen she chooses), resulting also in a fixed numerical date of any month, she has to insert (start) a dosage form. Secondly, a regimen of the subject invention also maintains or improves the suppression of follicular development due to the longer in-situ period of the dosage form and the shorter hormone-free period; in other words, a regimen of the subject invention maintains or in certain cases even improves ovarian suppression.

The subject invention also envisages a contraceptive kit for human female contraception which comprises at least two dosage forms, each dosage form to be used in one of two sequential cycles, and each dosage form to be started on numerical date ‘n+3’ of a month and removed on numerical date ‘n’ of the following month wherein ‘n’ is a numerical date of a month from 1-25.

The subject invention also envisages a contraceptive kit for human female contraception which comprises at least two dosage forms, each dosage form to be used in one of two sequential cycles, and each dosage form to be started on numerical date ‘m+4’ of a month and removed on numerical date ‘m’ of the following month wherein ‘m’ is a numerical date of a month from 1-24.

The subject invention further involves a contraceptive kit for human female contraception which comprises at least two dosage forms, each dosage form to be used in one of two sequential cycles, and each dosage form to be started on numerical date ‘y+5’ of a month and removed on numerical date ‘y’ of the following month, wherein ‘y’ is a numerical date of a month from 1-23.

The subject invention additionally provides a contraceptive kit for human female contraception which comprises at least two dosage forms, each dosage form to be used in one of two sequential cycles, and each dosage form to be started on numerical date ‘z+6’ of a month and removed on numerical date ‘z’ of the following month, wherein ‘z’ is a numerical date of a month from 1-22.

A contraceptive kit of the subject invention can be provided for any number of months starting with a kit for at least two cycles, i.e., a kit for two, three, four, five, six, etc. cycles. If the kit is for example for three months, then of course each dosage form is to be used in one of three sequential cycles; if the kit is for example for four months, then each dosage form is to be used in one of four cycles, etc.

The subject invention also provides a reminder system for a dosage regimen comprising choosing one particular numerical date of a month from 1-25, independent of the month, as the numerical date on which a dosage form is always removed and always starting a new dosage form three days later.

The subject invention further envisages a reminder system for a dosage regimen comprising choosing one particular numerical date of a month from 1-24, independent of the month, as the numerical date on which a dosage form is always removed and always starting a new dosage form four days later.

The subject invention additionally involves a reminder system for a dosage regimen comprising choosing one particular numerical date of a month from 1-23, independent of the month, as the numerical date on which a dosage form is always removed and always starting a new dosage form five days later.

The subject invention also provides a reminder system for a dosage regimen comprising choosing one particular numerical date of a month from 1-22, independent of the month, as the numerical date on which a dosage form is always removed and always starting a new dosage form six days later.

The subject invention also encompasses a contraceptive regimen for a dosage form of the subject invention wherein hormones are administered for a defined duration, characterized in that the cycle durations vary such as to correspond with the number of the days of the month in which the cycle was started. The defined duration can be any number of months starting with at least two months, i.e. two, three, four, five, six, etc. months. In a specific embodiment, the defined duration is three months.

The present invention is further described in the following examples which are not in any way intended to limit the scope of the invention as claimed.

### EXAMPLE 1

### Pharmacodynamic Trial: n, n+3 wherein n=1

An open-label randomized, comparative pharmacodynamic trial is carried out during the months February, March and April with a commercially available contraceptive vaginal ring (Nuvaring®) in a monthly regimen of the subject invention wherein the ring is inserted on each 4^{th} of the month (n+3) and then removed on the first (n) of the following month versus the standard 21/7 regimen. This trial is carried out in healthy female volunteers to assess the effects of the vaginal ring in this monthly regimen on ovarian function (pharmacodynamics) versus the effects on ovarian function in the standard 21/7 regimen.

Forty (40) healthy premenopausal women between 18 and 40 years of age at the time of screening participate in the trial for three treatment cycles.

The women are divided into two groups trial arm A and trial arm B. Trial arm A uses the vaginal ring following the standard regimen wherein the ring is worn for 21 days followed by a 7 day ring-free period. Trial arm B uses the vaginal ring in a regimen of the subject invention wherein the ring is inserted each 4^{th} day of the month and removed each first day of the following month.

Three times a week serum estradiol (E2), Progesterone (P), LH and FSH is measured and a transvaginal ultrasound scanning is carried out. A physical and gynecological examination is carried out at screening and at the end of treatment and cervical cytology is checked at screening.

### EXAMPLE 2

### Pharmacodynamic Trial m, m+4 wherein m=1

An open-label randomized, comparative pharmacodynamic trial essentially as described above in Example 1 is carried out in a monthly regimen of the subject invention wherein the ring is inserted on each 5^{th} of the month (m+4) and then removed on the first (m) of the following month versus the standard 21/7 regimen.

### EXAMPLE 3

### Pharmacodynamic Trial y, y+5 wherein y=1

An open-label randomized, comparative pharmacodynamic trial essentially as described above in Example 1 is carried out in a monthly regimen of the subject invention wherein the ring is inserted on each 6^{th} of the month (y+5) and then removed on the first (y) of the following month versus the standard 21/7 regimen.

### EXAMPLE 4

### Pharmacodynamic Trial z, z+6 wherein z=1

An open-label randomized, comparative pharmacodynamic trial essentially as described above in Example 1 is carried out in a monthly regimen of the subject invention wherein the ring is inserted on each 7^{th} of the month (z+6) and then removed on the first (z) of the following month versus the standard 21/7 regimen.

### EXAMPLE 5

### Exploratory Comparative Trial

An open label five-arm, randomized, group comparative, multicenter trial with the regimens as described in Examples 1, 2, 3 and 4 versus the standard 21/7 regimen is carried out with the same commercially available vaginal contraceptive ring to investigate different monthly regimens of the subject invention in healthy female volunteers. Contraceptive efficacy, vaginal bleeding characteristics, safety, compliance, and acceptability of these different monthly regimes is assessed compared to the standard 21/7 regimen.

Five hundred (500) healthy premenopausal women between 18 and 40 years of age at the time of screening participate in the trial for one year, i.e. for 12 months for the regimens of the subject invention or for 13 treatment cycles for the standard 21/7 regimen.

The women are divided into five (5) groups:

Trial arm A: standard regimen, 21 days of ring use, followed by a 7 days ring-free period;

Trial arm B: monthly regimen of the subject invention wherein the ring is inserted the 4^{th} of each month (n+3) and removed on the first of the following month (n); (3-4 days ring-free period);

Trial arm C: monthly regimen of the subject invention wherein the ring is inserted on the 5^{th} of each month (m+4) and removed on the 1^{st} of the following month (m) (4-5 days ring-free period);

Trial arm D: monthly regimen of the subject invention wherein the ring is inserted on the 6^{th} of each month (y+5) and removed on the 1^{st} of the following month (y) (5-6 days ring-free period);

Trial arm E: monthly regimen of the subject invention wherein the ring is inserted on the 7^{th} of each month (z+6) and removed on the 1^{st} of the following month (z) (6-7 days ring-free period).

Assessments are carried out at screening (within one month before starting treatment) and at 3, 6, 9 and 12 months or at premature discontinuation. At the screening visit, subjects provide medical and gynecological history and undergo a physical and gynecological examination, including cervical cytology. The physical and gynecological examinations are repeated at the last study visit. In addition, clinical safety laboratory tests are performed at screening and at the end of treatment. At all study visits, blood pressure and body weight is measured. A transvaginal ultrasound for assessment of endometrial thickness is performed at screening and repeated after one year. Endometrial biopsies are taken if the double layer endometrial thickness is 10 mm or more. Urinary pregnancy tests are performed by the subjects before the start of study treatment, at each study visit and if a pregnancy is suspected during the trial. The occurrence of adverse events and the use of concomitant medication are recorded throughout the trial. Vaginal bleeding patterns and compliance are recorded on diary cards.

### EXAMPLE 6

### Safety and Efficacy Trial n, n+3, wherein n=1

An open-label two-arm, randomized, group-comparative, multicenter trial is carried out to investigate contraceptive efficacy, vaginal bleeding characteristics, compliance, safety and acceptability with the same vaginal ring as used in Examples 1-5 in a monthly regimen of the subject invention wherein the ring is inserted each 4^{th} (n+3) of the month and removed each 1^{st} (n) of the following month versus the standard 21/7 regimen.

Thousand three-hundred and thirty (1330) healthy premenopausal women between 18 and 40 years of age at the time of screening participate in the trial for one year, i.e. for 12 months in the regimens of the subject invention or for 13 treatment cycles for the standard 21/7 regimen.

The women are divided into two (2) groups:

Trial arm A: 330 women participate in a standard regimen of 21 days of ring use, followed by a 7 days of ring-free (i.e. hormone-free) period;

Trial arm B: 1000 women participate in a monthly regimen of the subject invention wherein the ring is inserted on the 4^{th} of each month (n+3) and removed on the first of the following month (n) (3-4 days ring-free period);

Assessments are carried out at screening (within one month before starting treatment) and at 3, 6, 9 and 12 months or at premature discontinuation. At the screening visit, subjects provide medical and gynecological history and undergo a physical and gynecological examination, including cervical cytology. The physical and gynecological examinations and the cervical cytology are repeated at the last study visit. In addition, clinical safety laboratory test are performed at screening and at the end of treatment. At all study visits, blood pressure and body weight are measured. Urinary pregnancy test is performed by the subjects before the start of study treatment, at each study visit and if a pregnancy is suspected during the trial. The occurrence of adverse events and the use of concomitant medication is recorded throughout the trial. Vaginal bleeding patterns and compliance is recorded on diary cards.

The investigated regimen is found to result in very high compliance in comparison to the standard 21/7 regimen.

### EXAMPLE 7

### Safety and Efficacy Trial m, m+4 wherein m=1

An open-label two-arm, randomized, group-comparative, multicenter trial essentially as described in Example 6 is carried out to investigate contraceptive efficacy, vaginal bleeding characteristics, compliance, safety and acceptability with the same vaginal ring as used in Examples 1-5 in a monthly regimen of the subject invention wherein the ring is inserted each 5^{th} (m+4) of the month and removed the 1^{st} (m) of the following month versus the standard 21/7 regimen.

The investigated regimen is found to result in very high compliance in comparison to the standard 21/7 regimen.

### EXAMPLE 8

### Safety and Efficacy Trial y, y+5 wherein y=1

An open-label two-arm, randomized, group-comparative, multicenter trial essentially as described in Example 6 is carried out to investigate contraceptive efficacy, vaginal bleeding characteristics, compliance, safety and acceptability with the same vaginal ring as used in Examples 1-5 in a monthly regimen of the subject invention wherein the ring is inserted each 6^{th} (y+5) of the month and removed on the 1^{st} (y) of the following month versus the standard 21/7 regimen.

The investigated regimen is found to result in very high compliance in comparison to the standard 21/7 regimen.

### EXAMPLE 9

### Safety and Efficacy Trial z, z+6 wherein z=1

An open-label two-arm, randomized, group-comparative, multicenter trial essentially as described in Example 6 is carried out to investigate contraceptive efficacy, vaginal bleeding characteristics, compliance, safety and acceptability with the same vaginal ring as used in Examples 1-5 in a monthly regimen of the subject invention wherein the ring is inserted each 7^{th} (z+6) of the month and removed on the 1^{st} (z) of the following month versus the standard 21/7 regimen.

The investigated regimen is found to result in very high compliance in comparison to the standard 21/7 regimen.

1-36. (canceled)

37. A method of human female contraception which comprises starting a dosage form on numerical date ‘n+3’ of a month and removing the dosage form on numerical date ‘n’ of the following month, wherein ‘n’ is a numerical date of a month from 1-25 and wherein the method is repeatedly carried out for at least two cycles.

38. A method of human female contraception which comprises starting a dosage form on numerical date ‘m+4’ of a month and removing the dosage form on numerical date ‘m’ of the following month, wherein ‘m’ is a numerical date of a month from 1-24 and wherein the method is repeatedly carried out for at least two cycles.

39. A method of human female contraception which comprises starting a dosage form on numerical date ‘y+5’ of a month and removing the dosage form on numerical date ‘y’ of the following month, wherein ‘y’ is a numerical date of a month from 1-23 and wherein the method is repeatedly carried out for at least two cycles.

40. A method of human female contraception which comprises starting a dosage form on numerical date ‘z+6’ of a month and removing the dosage form on numerical date ‘z’ of the following month, wherein ‘z’ is a numerical date of a month from 1-22 and wherein the method is repeatedly carried out for at least two cycles.

41. The method according to claim 37 wherein the dosage form is a vaginal ring.

42. The method according to claim 37 wherein starting is vaginally administering.

43. The method according to claim 37 wherein the dosage form is a patch.

44. The method according to claim 37 wherein the dosage form comprises an estrogen and a progestogen.

45. The method according to claim 44 wherein the progestogen is etonogestrel and the estrogen is estradiol or a salt thereof or an ester thereof or the estrogen is ethinyl estradiol.

46. The method according to claim 44 wherein the progestogen is nomegestrol acetate and the estrogen is estradiol or a salt thereof or an ester thereof or the estrogen is ethinyl estradiol.

47. The method according to claim 41 wherein the dosage form is made of a thermoplastic material.

48. The method according to claim 41 wherein the dosage form comprises one or more compartments.

49. The method according to claim 48 wherein each compartment comprises one or more layers.

50. A contraceptive kit for human female contraception which comprises at least two dosage forms, each dosage form to be used in one of two sequential cycles and each dosage form to be started on numerical date ‘n+3’ of a month and removed on numerical date ‘n’ of the following month wherein ‘n’ is a numerical date of a month from 1-25.

51. A contraceptive kit for human female contraception which comprises at least two dosage forms, each dosage form to be used in one of two sequential cycles and each dosage form to be started on numerical date ‘m+4’ of a month and removed on numerical date ‘m’ of the following month wherein ‘m’ is a numerical date of a month from 1-24.

52. A contraceptive kit for human female contraception which comprises at least two dosage forms, each dosage form to be used in one of two sequential cycles and each dosage form to be started on numerical date ‘y+5’ of a month and removed on numerical date ‘y’ of the following month, wherein ‘y’ is a numerical date of a month from 1-23.

53. A contraceptive kit for human female contraception which comprises at least two dosage forms, each dosage form to be used in one of two sequential cycles and each dosage form to be started on numerical date ‘z+6’ of a month and removed on numerical date ‘z’ of the following month, wherein ‘z’ is a numerical date of a month from 1-22.

54. The contraceptive kit according to claim 50 which comprises three dosage forms.

55. The contraceptive kit according to claim 50 wherein the dosage form is a vaginal ring.

56. The contraceptive kit according to claim 50 wherein starting is vaginally administering.

57. The contraceptive kit according to claim 50 wherein the dosage form is a patch.

58. The contraceptive kit according to claim 50 wherein the dosage form comprises an estrogen and a progestogen.

59. The contraceptive kit according to claim 58 wherein the progestogen is etonogestrel and the estrogen is estradiol or a salt thereof or an ester thereof or the estrogen is ethinyl estradiol.

60. The contraceptive kit according to claim 58 wherein the progestogen is nomegestrol acetate and the estrogen is estradiol or a salt thereof or an ester thereof or the estrogen is ethinyl estradiol.

61. The contraceptive kit according to claim 55 wherein the dosage form is made of a thermoplastic material.

62. The contraceptive kit according to claim 55 wherein the dosage form comprises one or more compartments.

63. The contraceptive kit according to claim 62 wherein each compartment comprises one or more layers.

64. A reminder system for a dosage regimen comprising choosing one particular numerical date of a month from 1-25, independent of the month, as the numerical date on which a dosage form is always removed and starting a new dosage form three days later.

65. A reminder system for a dosage regimen comprising choosing one particular numerical date of a month from 1-24, independent of the month, as the numerical date on which a dosage form is always removed and starting a new dosage form four days later.

66. A reminder system for a dosage regimen comprising choosing one particular numerical date of a month from 1-23, independent of the month, as the numerical date on which a dosage form is always removed and starting a new dosage form five days later.

67. A reminder system for a dosage regimen comprising choosing one particular numerical date of a month from 1-22, independent of the month, as the numerical date on which a dosage form is always removed and starting a new dosage form six days later.

68. The reminder system according to claim 64 wherein the dosage form is a vaginal ring.

69. The reminder system according to claim 64 wherein starting is vaginally administering.

70. The reminder system according to claim 64 wherein the dosage form is a patch.

71. A contraceptive regimen wherein hormones are administered for a defined duration, characterized in that cycle duration varies such as to correspond with the number of the days of the month in which the cycle is started.

72. The contraceptive regimen according to claim 71 wherein a defined duration is at least three months.
